SEQUENCE LISTING (4 sequences)

SEQ ID NO: 1 - protein, 20 aa, Ananas comosus
VPQSIDWRDYGAVTSVKNQN

SEQ ID NO: 2 - protein, 20 aa, Ananas comosus
VPQSIDWRDSGAVTSVKNQG

SEQ ID NO: 3 - protein, 20 aa, Ananas comosus
VPQSIDWRNYGAVTSVKNQG

SEQ ID NO: 4 - protein, 21 aa, Unknown
  Amino terminal end of isolated polypeptide
AVPQSIDWRDYGAVTSVKNQN

(19) United States
(12) Patent Application Publication
(10) Pub. No.: US 2018/0264091 A1
(43) Pub. Date: Sep. 20, 2018

(54) ENZYMATIC FRACTIONS WITH ANTI-INFLAMMATORY ACTIVITY

(71) Applicant: Anatara Lifesciences Limited, Brisbane (AU)

(72) Inventors: Tracey L. Mynott, Brisbane (AU); Christian Engwerda, Brisbane (AU)

(21) Appl. No.: 15/753,948

(22) PCT Filed: Aug. 18, 2016
(86) PCT No.: PCT/US2016/047523

§ 371 Date: Feb. 20, 2018

Related U.S. Application Data
(60) Provisional application No. 62/207,570, filed on Aug. 20, 2015.

(51) Int. Cl. A61K 38/48; A61P 37/06

(52) U.S. Cl. CPC A61K 38/4873; A61P 37/06

(57) ABSTRACT

Certain components of crude bromelain extract have surprisingly been found to not support, or to interfere with, the anti-inflammatory benefit of bromelain. By removing the detrimental components, the remaining component(s) have increased anti-inflammatory effectiveness.

### RELATED APPLICATIONS

This application asserts priority from U.S. provisional patent filing Ser. No. 62/207570, filed 20 Aug. 2015, the contents of which are here incorporated by reference.

### GOVERNMENT INTEREST

None

### PARTIES TO JOINT RESEARCH AGREEMENTS

None applicable.

### SEQUENCE LISTING

This application incorporates by reference the machine-readable sequence listing files submitted here.

### PRIOR DISCLOSURES BY THE INVENTOR

None.

### BACKGROUND

Pineapples have a long tradition as a medicinal plant among the natives of South and Central America. The first isolation of bromelain was recorded by the Venezuelan chemist Vicente Marcano in 1891 from the fruit of pineapple. In 1892, Russell Henry Chittenden, assisted by Elliott P. Joslin and Frank Sherman Meara, investigated the matter fully, and called it ‘bromelin’. Later, the term “bromelain” was introduced, and originally the term was applied to any protease from any member of the plant family Bromeliaceae.

Bromelain has a long history of folk and modern medicinal use and continues to be explored as a potential healing agent in alternative medicine. It is also widely accepted as a phytotherapeutical drug. Bromelain was first introduced as a therapeutic supplement in 1957. First, research on bromelain was conducted in Hawaii, but more recently has been conducted in countries in Asia, Europe, and Latin America. Recently, researchers in Germany have taken a great interest in bromelain research. Currently, bromelain is the thirteenth most widely used herbal medicine in Germany.

Some of the therapeutic benefits of bromelain are reversible inhibition of platelet aggregation, reversible inhibition of angina pectoris, reversible inhibition of bronchitis and sinusitis, treating surgical traumas, thrombophlebitis, and pyelonephritis. It can also be used after surgery or injury to reduce swelling (inflammation), especially of the nose and sinuses. It is also used for preventing muscle soreness after intense exercise. Bromelain also has been reported to interfere with the growth of tumor cells and slow blood clotting. Bromelain is also used for hay fever, treating a bowel condition that includes swelling and ulcers (ulcerative colitis), removing dead and damaged tissue after burns (debridement), preventing the collection of water in the lung (pulmonary edema), relaxing muscles, stimulating muscle contractions, improving the absorption of antibiotics, preventing cancer, shortening labor, and helping the body get rid of fat. In food preparation, bromelain is used as a meat tenderizer, and to clarify beer.

Systemic enzyme therapy (consisting of combinations of proteolytic enzymes such as bromelain, trypsin, chymotrypsin, and papain) has been investigated in Europe for the treatment of breast, colorectal, and plasmacytoma cancer patients. In mice with experimental colitis, six months of dietary bromelain from pineapple stem or from fresh juice decreased the severity of colonic inflammation and reduced the number of cancerous lesions in the colon.

As a potential anti-inflammatory agent, bromelain may be useful for treating arthritis, allergic airway disease and multiple sclerosis but has neither been confirmed in human studies for this use, nor is it approved with a health claim for such an effect by the Food and Drug Administration or European Food Safety Authority. The Natural Medicines Comprehensive Database suggests that bromelain, when used in conjunction with trypsin (a different protease) and rutin (a substance found in buckwheat) is as effective as some prescription analgesics in the management of osteoarthritis. A product (WOBENZYME™) that combines bromelain with trypsin and rutin is available commercially and seems to reduce pain and improve knee function in people with osteoarthritis. However, the National Institutes of Health notes, “There isn't enough scientific evidence to determine whether or not bromelain is effective for any of its other uses.”

The art teaches to use the whole crude extract of bromelain. I have, however, surprisingly found that crude bromelain includes components that are detrimental to the desired effectiveness of the crude bromelain. I have thus separated bromelain crude extract into a number of distinct fractions, each containing specific enzymes and other components. I have then methodically tested each of these fractions, to identify which fractions provide which effects. I have thus found a way to improve the usefulness of bromelain crude extract by separating it into distinct fractions, each of which is appropriate for certain specific uses.

### BRIEF DESCRIPTION OF THE FIGURES

FIG. 1 provides a schematic flow chart of a process for separating bromelain into various component fractions.

FIG. 2 shows A) the ion exchange chromatography spectrum for bromelain extract, identifying Fraction 2 (F2) and Fraction 3 (F3) and peaks CCY, CCW and CCS; and B) the major protein species identified by SDS-PAGE of the CCY, CCW and CCS fractions of the crude extract.

FIG. 3 shows a size-exclusion chromatography profile and metal affinity chromatography profile of the CCS fraction to produce pure ananain (A, B) and the size of the ananain protein by SDS-PAGE (C).

FIG. 4 shows a comparison of the amino-terminal ends of certain of the polypeptides isolated here.

FIG. 5—Effect of ananain on cytokine levels in mice at 2 or 6 hrs post anti-CD3 mAb administration.

FIG. 6—Effect of bromelain on cytokine levels in mice at 2 or 6 hrs post anti-CD3 mAb administration.

FIG. 7—Effect of F1 on cytokine levels in mice administered at 2 or 6 hrs post mAb (+mAb) or no mAb antibody (−mAb) administration.

### DETAILED DESCRIPTION:

### Bromelain and Its Components

“Bromelain” is the collective name for a crude proteolytic extract obtained from the pineapple plant (Ananas comosus). Two forms of bromelain are known; fruit bromelain obtained from fresh pineapple fruit, and stem bromelain obtained from the stem of the plant. The main commercial source of bromelain is stem bromelain; thus, the terms “bromelain” and “stem bromelain” are often used interchangeably.

### Stem Bromelain

The major proteolytic enzyme within bromelain is a protease called stem bromelain, CAS 37189-34-7 (EC 3.4.22.32). This protease enzyme is referred to as a sulfhydryl protease, since a free sulfhydryl group of a cysteine side-chain is required for function. Stem bromelain has a broad specificity for cleavage of proteins, and has a strong preference for Z-Arg-Arg-|-NHMec among small molecule substrates.

### Fruit Bromelain

The major protease within the fruit bromelain extract is called fruit bromelain (EC 3.4.22.33). This protease enzyme is referred to as a sulfhydryl protease, since a free sulfhydryl group of a cysteine side-chain is required for function. Fruit bromelain has a strong preference for Bz-Phe-Val-Arg-|-NHMec.

### Ananain

Ananain (EC 3.4.22.31) may be isolated from the stem of the pineapple plant. It differs from stem and fruit bromelains in being inhibited by chicken cystatin. It catalyzes the hydrolysis of proteins, with a broad specificity for peptide bonds. The best reported small molecule substrate is Bz-Phe-Val-Arg-|-NHMec, albeit ananain has broader specificity than fruit bromelain.

### Comosain

Comosain may be isolated from the stem of the pineapple plant. The best reported small molecule substrate for comosain is Z-Arg-Arg-NH-Mec. It has an N-terminal twenty amino acid sequence, VPQSI DWRNY GAVTS YKNQG. It is homologous to ananain save for one residue.

### Manufacture of CCS, CCW and CCY and F2 and F3

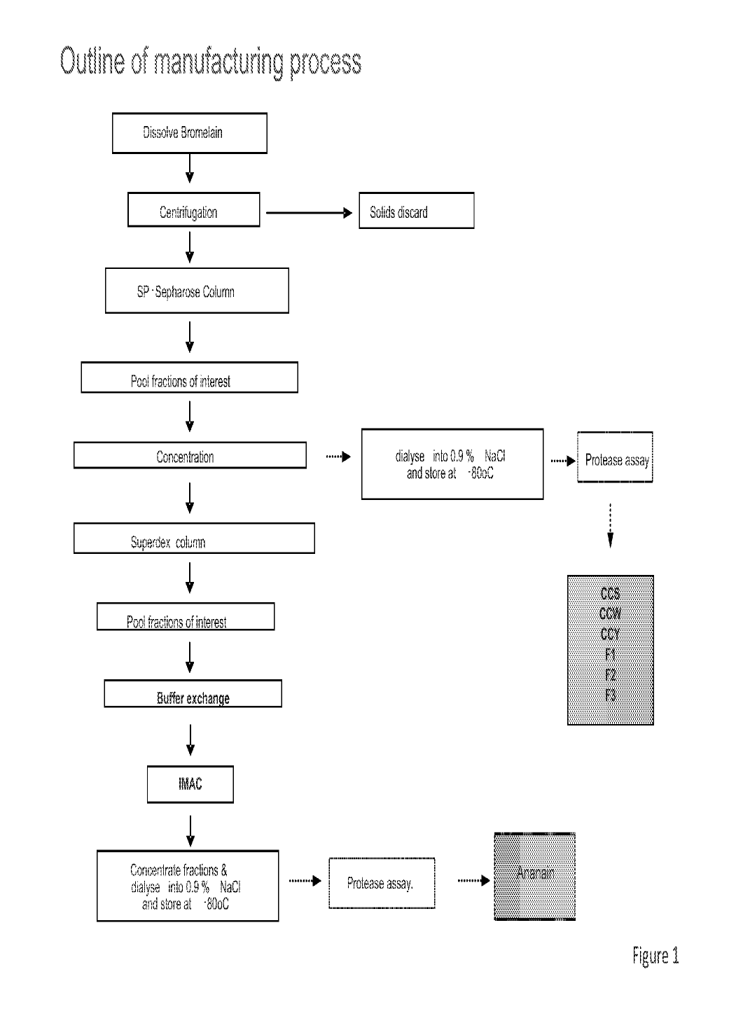

A flow chart outlining an exemplary manufacturing process is shown in FIG. 1.

### Reagents

Crude bromelain (proteolytic activity, 1,541 nmol/min/mg) was obtained from Solvay Inc. (Germany). SP-Sepharose HP was from GE Healthcare (Sydney, Australia). Superdex 75 media and Hi-Trap Metal Affinity FF chelating Sepharose was from Amersham Biosciences. Precast 4-12% Bis-Tris NuPAGE acrylamide gels were from Invitrogen (Sydney, Australia). Broad range molecular weight markers were from Bio-Rad Laboratories (Sydney, Australia), and polyvinylidene fluoride (PVDF) western blotting membranes were from Roche Diagnostics GmbH (Castle Hill, Australia). Precast 12% acrylamide mini gels were from Novex-Electrophoresis (Frankfurt, Germany). Pharmalyte 3˜10™, Ampholine 9-11, Ready Mix IEF (acrylamide, bisacrylamide) and LEF markers were obtained from Pharmacia Biotech. All other reagents were of analytical grade and obtained from Sigma Chemical Co.

### Preparation of Bromelain

All the following steps were performed at ambient temperature (20 to 25° C.). A solution of bromelain (30 mg/ml) was prepared by dissolving 450 mg of bromelain powder in 15 ml of 20 mM acetate buffer (pH 5.0) containing 0.1 mM EDTA, sodium. The solution was then centrifuged at 13,000×g for 10 minutes to remove insoluble material. The clear supernatants were used for chromatography.

### Separation by Ion Exchange Chromatography

A Fast flow S-sepharose column was prepared by packing 25 ml of media into an XK 16/20™ column (Pharmacia Biotech) and equilibrated with 20 mM acetate buffer (pH 5.0) containing 0.1 mM EDTA on an FPLC system at 3 ml/min. 5 ml of bromelain solution was injected onto the column. Unbound protein was collected and the column washed with 100 ml of acetate buffer. Protein bound to the column was eluted with a linear gradient of 0 to 0.8 M NaCl in acetate buffer over 300 ml. Five (5) ml fractions were collected throughout the gradient.

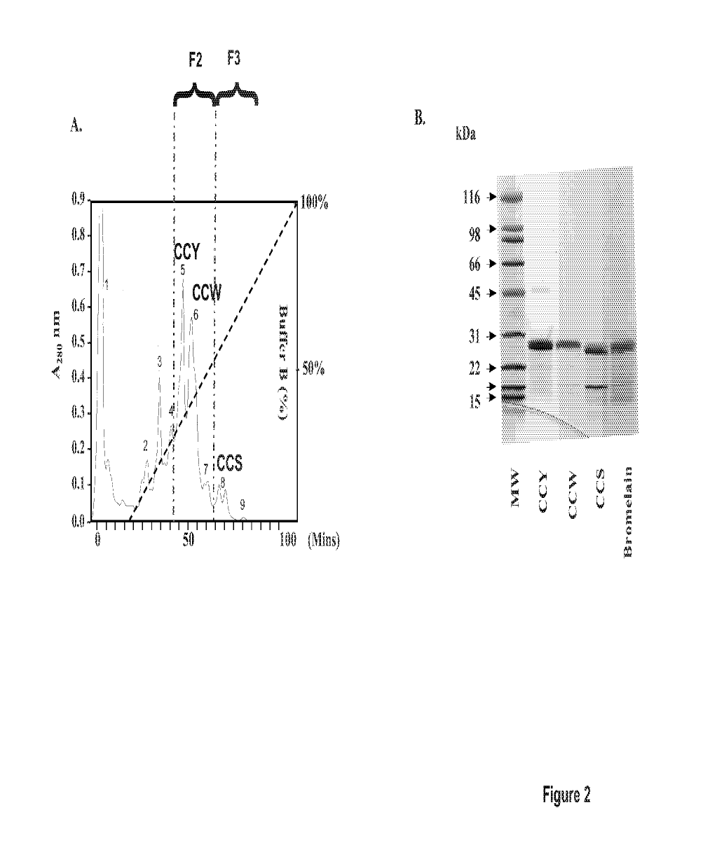

FIG. 2A shows a typical U.V. chromatogram of the various peaks obtained from crude bromelain obtained from this procedure.

The CCY, CCW, CCS peaks or the F2 and F3 fractions of interest identified from the U.V. profile were pooled and concentrated by ultrafiltration using a filtron stirred cell containing an ultrafiltration membrane of nominal molecular weight cut-off of 10 kDa. The fractions were then buffer exchanged using PD10 columns (Pharmacia Biotech) into isotonic saline (0.9 % w/v NaCl), sterile filtered (0.2 um) and adjusted for protein content or proteolytic activity. Samples were then frozen at −80° C. until required.

FIG. 2B shows the SDS-PAGE of peaks which I term “CCW” and “CCY” (which contain stem bromelain protease), and peak “CCS” (which contains ananain).

The CCY peak elutes as the 5^{th}main protein peak from the column (30% of Buffer B, at 45 to 50 minutes), while CCW elutes as the 6^{th} major protein peak (35% Buffer B; at 50 to 57 mins). CCS is the last double peak (or peak 8) off the column, eluting at 65 to 75 minutes (60% Buffer B).

The F2 fraction comprises of peak 5 (CCY), peak 6 (CCW) as well as peak 7 and is collected as one fraction eluting from 45 to 65 minutes (35%) to 60%) Buffer B). The F3 fraction comprises the CCS peak as well as trailing peak 9 and is collected as one fraction eluting at 65 to 85 minutes (60 to 80% Buffer B).

| Description | Anti-secretory active | Anti-inflammatory active |
|---|---|---|
| Fraction | F2 | F3 |
| Peak | CCY & CCW | CCS |
| Pure API | Stem bromelain protease | ananain |

Products can be developed using either pure API, or cruder forms and combinations of F2 and F3. Very effective anti-diarrhea agents may include
Pharmaceutical product—Stem bromelain protease combined with ananain—pure entities.
Less regulated product. CC series—partially pure fraction CCY, CCW and CCS
Less regulated product. F series—crude fractions. F2&F3 fraction of bromelain (anti-secretory and anti-inflammatory tractions of bromelain, minus the pro-inflammatory component).
Products may be derived from the natural source (i.e. from bromelain), or expressed in a pichia (yeast) expression system.
Products with a higher ratio of F3:F2 (i.e. more of the anti-inflammatory active) are more likely to be effective than the ratio found in natural bromelain.

a) Protein Assay:

Protein concentrations were determined using a BCA™ Protein Assay kit (Pierce, Rockford, USA). Samples were compared to bovine serum albumin standards (0 to 1.5 mg/ml) prepared in either 0.9% saline or 20 mM acetate buffer pH 5.0, as appropriate.

b) Proteinase Assay:

Specific activity of the various chromatographic fractions and of the pure proteases was determined by monitoring the release of p-nitroaniline from the peptide-p-nitroanilide (pNA) substrate Z-Arg-Arg-pNA (Bachem, Saffron Walden, UK) as described by Napper et al in Biochem. J., 301, 727-735, (1994). This assay was based on that described by Filippova et al in Anal. Biochem., 143, 293-297 (1984). All assays were performed in 96 well plates at 30° C. using an iEMS kinetic plate reader (Life Sciences International, Basingstoke, UK). The maximum rate of reaction was determined by Genesys™ software (Life Sciences International, Basingstoke, UK) and the results expressed as nmoles/min of substrate converted.

c) Sodium Dodecyl Sulphate Polyacrylamide Gel Electrophoresis (SDS-PAGE):

Samples were analysed by SDS-PAGE on precast 4 to 20% T gradient gels. Samples were dissolved in 300 ul of SDS-PAGE sample buffer (62.5 mM Tris-HCl pH 6.8 containing 10% v/v glycerol, 2% w/v sodium dodecyl sulphate and 40 mM dithiothreitol) and heated at 95° C. in a water bath.

SDS-PAGE broad range molecular weight standards diluted 1:20 in SDS-PAGE sample buffer were treated similarly and run with the samples. Gels were ran on a mini Protean IITM electrophoresis system according to Bio-Rad's protocol at 240 V and until the dye front reached the end of the gel (30 to 45 min).

After electrophoresis, separated proteins were stained overnight with orbital mixing in a solution of 0.075% w/v colloidal brilliant blue G-250 containing 1.5% v/v phosphoric acid, 11.25% w/v ammonium sulphate and 25% v/v methanol. Gels were de-stained, to obtain a clear background, in a solution of 25% v/v methanol and 10% v/v acetic acid.

### Separation by SEC and IMAC

To isolate ananain, the “CCS” peak from the fractionation protocol was isolated used for further processing to obtain pure ananain using Size Exclusion Chromatography and Immobilised Metal Affinity Chromatography.

Size Exclusion Chromatography. The CCS peak was applied to a Superdex 75 HR 10/30 column pre-equilibrated with Buffer B (0.1 M sodium acetate, 1 mM di-sodium EDTA, 0.25 M NaCl, pH 5.0), and components were eluted at a flow rate of 1 ml/min. Fractions (0.5 ml) were collected, and those associated with absorbance peaks were pooled. These pools were analysed for specific activity against the Z-Arg-Arg-pNA substrate.

Immobilised Metal Affinity Chromatography (IMAC). The last eluting peak was buffer exchanged and concentrated by dia-filtration (spin concentrator of 5,000 da nominal molecular weight cut-off) into Buffer C (20 mM sodium phosphate, 1 M NaCl, pH 7). A Hi-Trap Metal Affinity Column (1 ml) was used to further separate components and was prepared as follow: first the column was loaded with Cu²⁺ (0.5 ml of 0.1 M copper sulphate), washed with 10 column volumes of Buffer D (20 mM sodium phosphate, 1 M NaCl, 2 M NH₄Cl, pH 7), then equilibrated with Buffer C. Concentrated protein samples (5 mg/ml) were then applied to the column at a flow rate of 1 ml/min and bound components were eluted by a 0-2 M linear gradient of NH₄Cl. Fractions (1 ml) were collected into tubes containing Buffer E (0.2 M sodium acetate, 1 mM di-sodium EDTA, pH 5, 0.5 ml). Fractions were pooled according to absorbance peaks. Each peak was then buffer exchanged into isotonic saline (0.9% w/v NaCl). All peaks were assayed for specific activity.

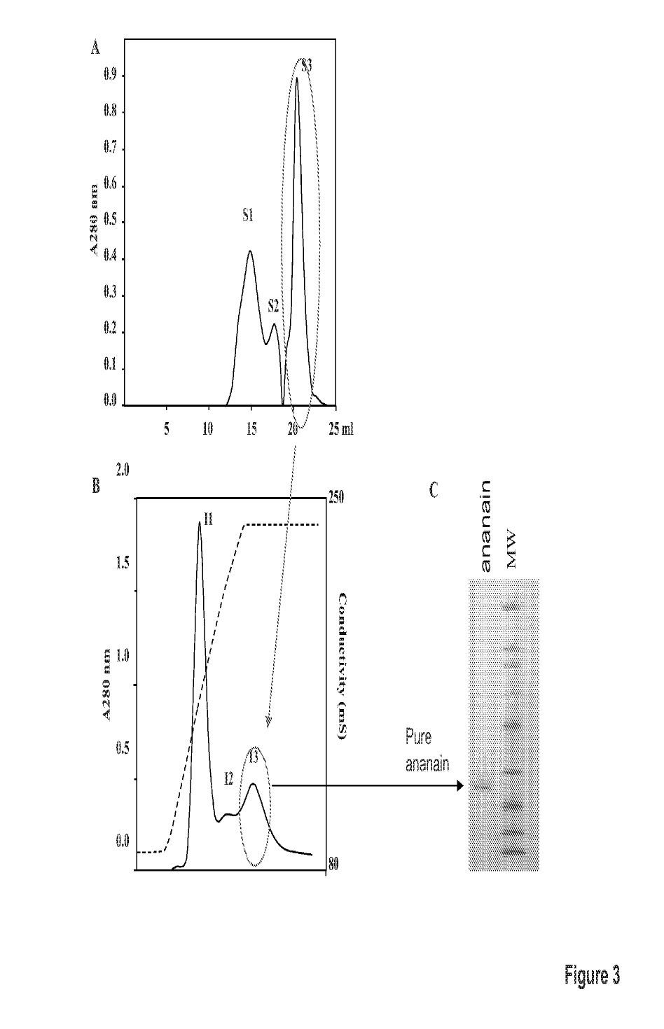

FIG. 3A shows a typical U.V. chromatogram of the CCS peak following size exclusion chromatography. FIGS. 3B and C shows the chromatogram of ananain obtained from IMAC and the purity as observed by SDS-PAGE.

### Confirming Identity

a) Isoelectric Focusing

Samples (0.5 to 1.0 mg/ml) were diluted 1:3 with deionised water and run on gradient gels of pH 3 to 11. Gels were cast using Ready Mix IEF™ to produce a 5.5% T, 3% C polyacrylamide gel containing 10% v/v glycerol, 5.0% Pharmalyte 3—1 OTM and 2.5% Ampholine 9˜11 TM. Briefly, 10 ul of sample and high pI markers were loaded onto the gel after prefocusing at 700 V. Sample entry was at 500 V for 10 min, focusing was at 2500 V for 1.5 hour and band sharpening at 3000 V for 10 min. After electrophoresis the proteins were fixed with a solution of 20% w/v TCA for 30 min, washed in destain for 30 min to remove TCA and stained with brilliant blue G-250 as described for SDS-PAGE (see above).

b) Protein Sequencing

To confirm identity of the isolated polypeptides, I sequenced the amino-terminal ends. For NH₂-terminal sequencing, proteins separated by SDS-PAGE were electroblotted to PVDF membrane, stained with 0.025% (w/v) Coomassie blue R-250 in 40% (v/v) methanol and de-stained in 50% (v/v) methanol. The membrane was then air-dried and proteins sequenced by automated Edman degradation using a gas phase sequencer (Applied Biosystems, Foster City, USA), equipped with an on-line phenylthiohydantion amino acid analyser.

### Results

A summary of the components obtained following chromatography, their calculated molecular weights, proteolytic activity against the synthetic peptide Z-Arg-Arg-pNA, and isoelectric point is shown in Table 1.

TABLE 1
Summary of molecular weight, pI, and specific activity of fractions purified from
bromelain

| | | Major protein | MW major | | Z-Arg-Arg-pNA |
|---|---|---|---|---|---|
| Column | Peak | species | species (kDa) | pI | (nmol/min/mg) |
| Ion exchange | CCY | Stem bromelain | 26 | 9.6, 9.7 | 1322 |
| | | (39) | | | |
| | CCW | Stem bromelain | 26 | 9.57, 9.6, 9.7 | 1092 |
| | | (39) | | | |
| | CCS | Ananain, (16, 40); | 24 | 10.4, 10.45 | 460 |
| | | comosain (24) | 26 | | |
| Superdex | S1 | Comosain | | | 491 |
| | | Ananain | | | |
| | S2 | Comosain | | | 332 |
| | | Ananain | | | |
| | S3 | Ananain | | | 70 |
| | | Comosain | | | |
| IMAC | I1 | Ananain | 24 | | 53 |
| | I2 | Ananain | 24 | | 502 |
| | I3 | Ananain | 24 | | 59 |

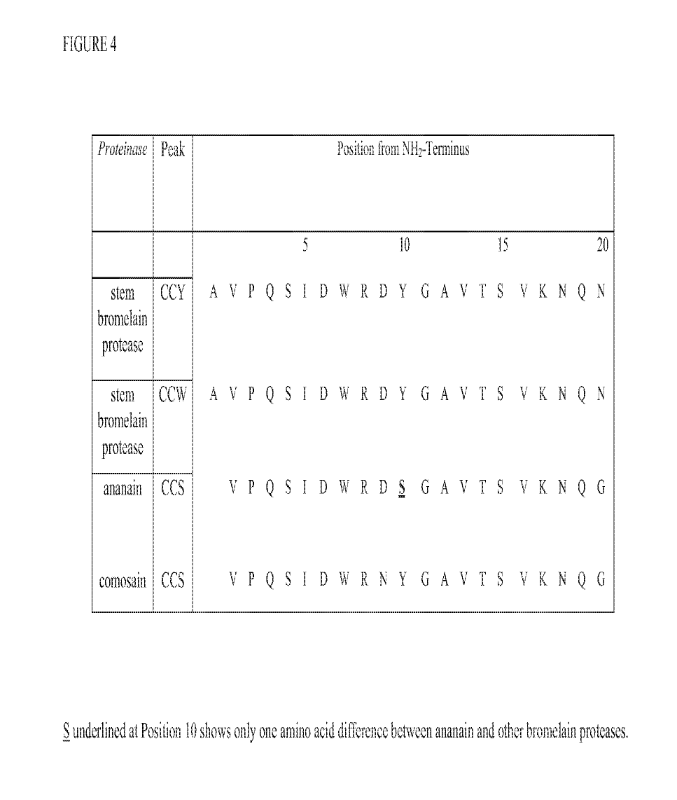

FIG. 4 compares the first 21 NH2-terminal amino acids of stem bromelain protease from CCY and CCW, ananain and comosain from CCS. Note the S underlined at Position 10 shows only one amino acid difference exists between ananain and other bromelain proteases. FIG. 4 shows that all proteins share sequence homologies. Ananain and comosain differ by two out of twenty amino acids when compared to stem bromelain protease. Comosain differs by two amino acids from ananain. Whilst it is clear that these proteins are structurally related, they are all distinct, showing divergence from each other.

### Different Fractions Have Different Activities

### Different Fractions of Bromelain have Different Biological Activities

### 1. Background

From the prior art, it is clear that bromelain has a variety of opposing physiological effects. Sometimes it acts to stimulate the immune system, while sometimes it inhibits the immune system. This is, of course, is a major disadvantage if the bromelain to be administered to induce one type of effect, induces the complete opposite and unwanted effect.

It would therefore be beneficial if individual components of bromelain giving rise to unwanted effects could be removed so as to lessen the possibility of side effects. We have now identified active fractions of crude bromelain which are responsible for the varied biological effects. Although not single proteins, these fractions comprise of only a few components and so the possibility of side effects when they are administered to patients is greatly reduced compared with crude bromelain.

### ii. Study Outline

In this series of studies, we used a model of immune activation to investigate the biological effects of bromelain and its components. Here, a monoclonal antibody (mAb), called anti-CDSε mAb binds to CD3 (the T cell receptor) on T cells and natural killer (NK)-T cells. This binding of the T cell receptor then activates the T cells and other immune cells and induces a systemic release of cytokines, including tumor necrosis factor (TNF) and interferon (IFN)γ, as well as other pro-inflammatory cytokines. Elevated levels of pro-inflammatory cytokines, such as TNF induces self-limited diarrhea in mice and humans.

a. Fractionation of Bromelain

Bromelain was fractionated by column chromatography to produce the F1, F2 and F3fractions as described above, except at much larger scale. That is, 20 g of natural bromelain was loaded onto an XK30/50 column to produce much larger quantities of materials. The F1, F2, F3 fractions were buffered exchanged by membrane dialysis.

The column retained approximately 66% of the total protein loaded onto it; the volume and protein content of each fraction eluted from the column was;
F1: 980 mL @4.1 mg/ml=4.018 mg
F2: 1270 mL @6.2 mg/ml=7.874 mg
F3: 875 mL @1.6 mg/ml=1.400 mg

A subsample of F3 was further fractionated to produce ananain.

A further subsample of F2 (12.70 mL) and F3 (8.75 mL) were re-combined to produce F2&F3 in the same proportion (85:15) found in the 66% of bromelain polypeptides remaining after ion exchange chromatography.

In a similar experiment, a farther subsample of F2 (1 mL) and F3 (4 mL) were re-combined to produce a new F2&F3 mixture with a higher amount of F3 present.

The “F2&F3” mixture thus produced compares to the naturally-occurring mixture as follows:

| Description | F2 | F3 |
|---|---|---|
| Natural bromelain ratio | 3.80 | 1 |
| Ion exchange | 2.30 | 1 |
| New F2&F3 mixture | 0.23 | 1 |

As noted above, fruit bromelain has a strong preference for Bz-Phe-Val-Arg-|-NHMec. By removing the F1 fraction, we expect to minimize Bz-Phe-Val-Arg-|-NHMec digestion.

These fractions were then investigated for immune activity.

b. Anti-CD3ε Model of In Vivo T Cell Stimulation.

Briefly, 6 to 10 week old C57BL6 female mice were administered bromelain, F1, F2, F3, combined F2 and F3, and ananain by oral gavage (40 mg/kg at 10 ml/kg). Two hours later, mice were then administered 0.3 μg (0.015 mg/kg) of anti-CD3ε mAb (iv) at a rate of 200 μl/mouse (10 ml/kg) in 0.9% pyrogen free saline.

Orbital blood samples were collected from mice two hours post antibody administration. Terminal blood samples were collected via cardiac puncture six hours post antibody administration. Two time points were assessed for cytokine levels, as some cytokines are rapidly produced in serum (eg. TNF, IL-2 and IL4), while other cytokines (eg, IFNγ) are delayed. Serum was collected from clotted blood samples and assayed for serum cytokine levels (pg/mL) using a Th1/Th2 cytokine kit (BD) and FACS analysis.

c. Assessment of Cytokine Levels.

The serum cytokine concentration was quantified using a Th1/Th2 Cytokine Cytometric Bead Array Analysis as per the manufacturer's (BD Biosciences, North Ryde, Australia) recommendations.

d. Statistical Analysis.

Statistical comparisons were made using the unpaired Student's t test, or one-way ANOVA with the Dunnett post-hoc test, where appropriate. In each group, values from all mice were included.

### iii. Results

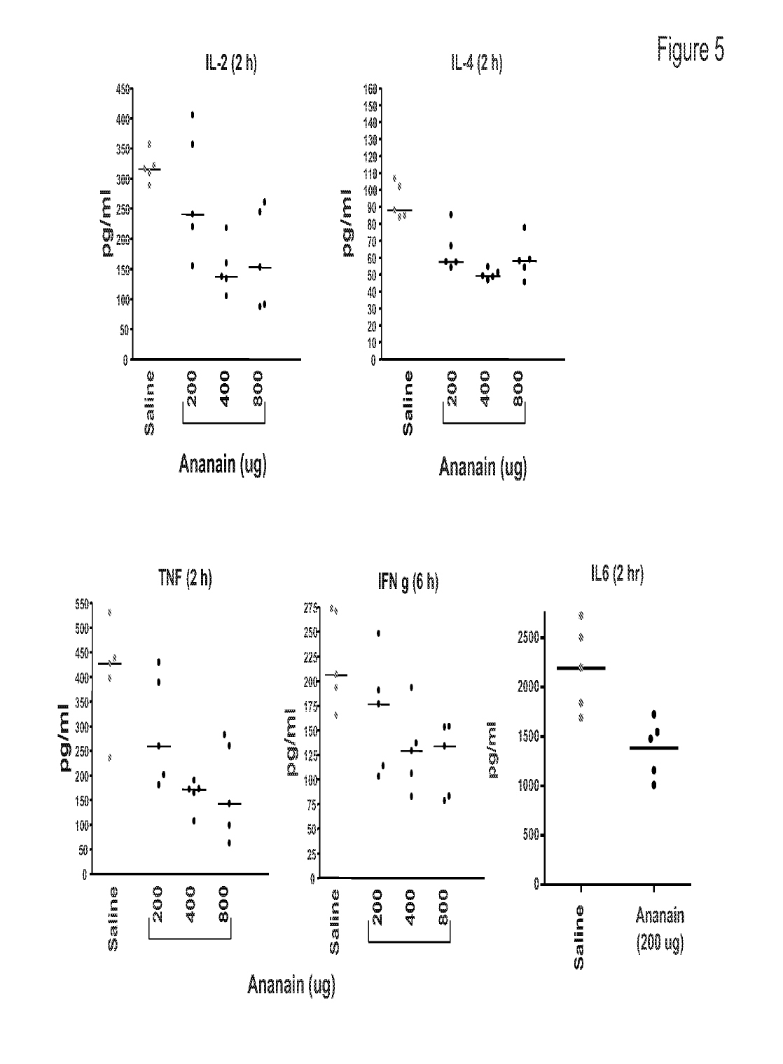

FIGS. 5 to 7 show the results of the effect of ananain, bromelain and F1 on cytokine levels. FIG. 5—Effect of ananain on cytokine levels in mice at 2 or 6 hrs post anti-CD3 mAb administration.

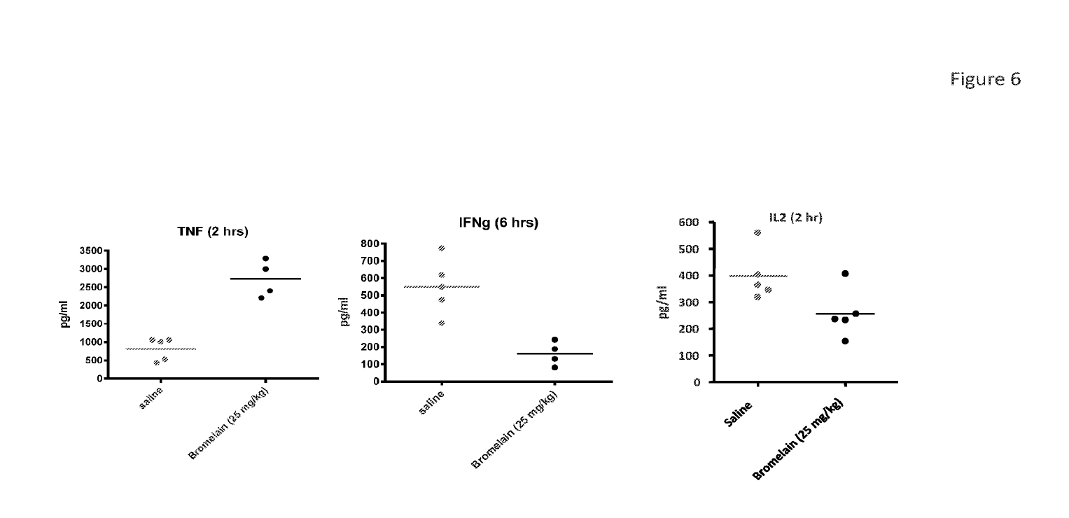

FIG. 6—Effect of bromelain on cytokine levels in mice at 2 or 6 hrs post anti-CD3 mAb administration.

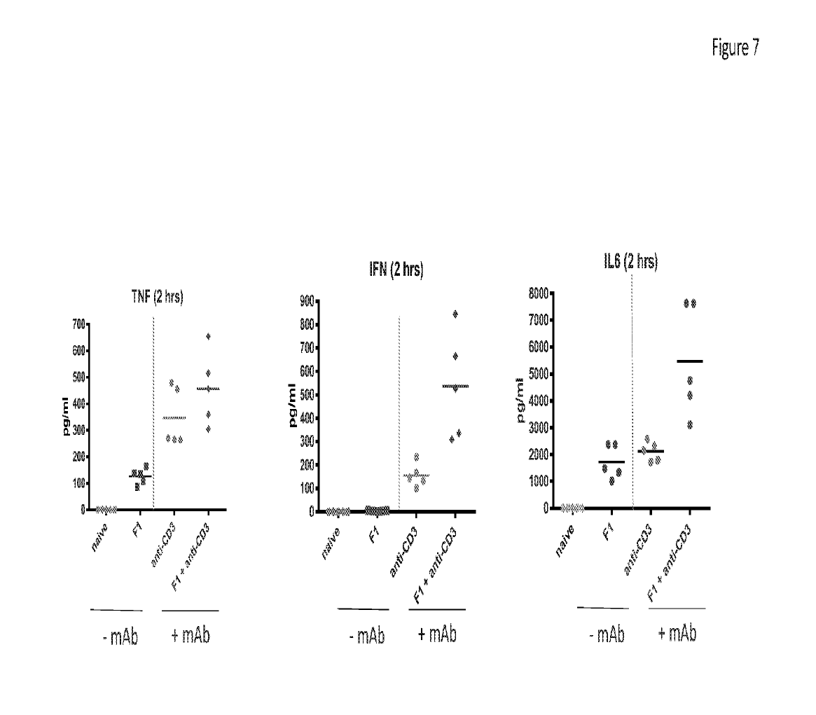

FIG. 7—Effect of F1 on cytokine levels in mice administered at 2 or 6 hrs post mAb (+mAb) or no mAb antibody (−mAb) administration.

### Effect of Ananain on Cytokine Levels In Vivo

We have previously described the immunosuppressive effects of ananain (patents derived from Mynott et al., PCT/GB98/00590). So we first confirmed that ananain would inhibit cytokine production in this model of immune activation.

FIG. 5 shows that a single oral dose of ananain dose dependency blocks the production of several different cytokines, including interleukin (IL)-2 (p=0.0037). IL-4 (p<0.0001), as well as TNF (p=0.002) and IFNγ (p=0.0153). Ananain at the lowest dose level (200 ug) also inhibited IL-6 production. These data confirm that ananain is an anti-inflammatory agent.

### Effect of Bromelain on Cytokine Levels In Vivo

FIG. 6 shows that bromelain has a different effect on cytokine levels than ananain. Like ananain, bromelain did decrease IL-2 (p<0.05) and IFNγ (p<0.04) production. However, in contrast to ananain, bromelain potently stimulated TNF, and IL-6 production (IL-6 data not shown).

### TNF and IL-6 are Potent Pro-inflammatory Cytokines.

The primary role of TNF is to regulate immune cells. TNF can induce fever, cachexia (body wasting), and inflammation. Increased levels of TNF production has been implicated in a variety of human diseases including major depression and inflammatory bowel disease (IBD).

IL-6 stimulates the inflammatory and auto-immune processes in many diseases such as diabetes, atherosclerosis, depression, systemic lupus erythematosus, and rheumatoid arthritis.

Therefore, despite bromelain's ability to block MAP kinase signaling and decrease cytokine levels in vitro (Mynott et al., 1999) it has a different, and often opposing, effect on cytokine production to ananain in vivo. Ananain does consistently inhibit cytokine production in vivo, however, bromelain can both simultaneously stimulate and inhibit cytokine production in vivo.

### Effect of F3 on Cytokine Levels In Vivo

Since ananain is derived from the F3 fraction of bromelain, we next investigated its immunosuppressive effects in the mouse model of immune activation.

Table 2 shows that a single oral dose of F3, like ananain blocks the production of several different cytokines, including IL-2, IL-4, and TNF.

TABLE 2
Effect of F3 on cytokine levels in mice administered anti-CD3 mAb

| | TNF (2 hrs) | IL-2 (2 hrs) | IL-4 (2 hrs) |
|---|---|---|---|
| Saline | 261 ± 20.5 | 398 ± 43 | 48.3 ± 5.3 |
| F2 | 187 ± 13.5 | 260 ± 41 | 36.1 ± 5.2 |
| % reduction | 28%* | 35%* | 25% |

*p < 0.05

### Effect of F2 on Cytokine Levels In Vivo

Table 3 shows that the F2 fraction of bromelain has negligible effect on cytokine production in this mouse model of immune activation. This fraction does not contain the immunosuppressive component, ananain.

TABLE 3
Effect of F2 on cytokine levels in mice administered anti-CD3 mAb

| | TNF (2 hrs) | IFNγ (6 hrs) | IL-4 (2 hrs) | IL-6 |
|---|---|---|---|---|
| Saline | 236 ± 17 | 269 ± 56 | 48.3 ± 5.3 | 839 ± 123.9 |
| F2 | 259 ± 16 | 220 ± 32 | 44.4 ± 3.8 | 923.6 ± 103.3 |
| % change | <10% | 18% | <10% | 10% |

### Effect of F1 on Cytokine Levels In Vivo

FIG. 7 shows that the F1 extract of bromelain stimulates cytokine production. This immunostimulatory action was not observed in the F2 or F3 fractions of bromelain. F1 potently stimulates the production of IFNγ at 2 hours post anti-CD3 mAb administration, when IFNγ is not normally observed at these high levels until at least 6 hours after antibody administration (see FIG. 5 and FIG. 6). We also observed that F1 alone stimulates cytokine production in mice not administered anti-CD3 mAb (p<0.02). That is, not only does F1 increases cytokine levels in an already stimulated immune system (i.e. where anti-CD3 is used to stimulate immune responses), but that F1 acts as an immunostimulant in its own right.

The presence of F1 in the bromelain sample may therefore be problematic if the purpose of administering bromelain was to suppress immune responses. That is, the presence of F1 would have an unwanted effect to further stimulate or exacerbate immune responses.

### Effect of Combined F2 and F3 on Cytokine Levels In Vivo

We next investigated the effect of bromelain, minus the F1 fraction, on cytokine production.

Table 4 shows that at 2 hours post antibody administration, combined F2&F3 (p< 0.05) decreased IL-2 levels, and had a moderate inhibitory effect on IFNγ. Earlier we saw that bromelain also reduced IL-2 and IFNγ (FIG. 6). However, unlike bromelain, combined F2/F3did not stimulate TNF production. This was because the immune stimulatory component F1, that had potent ability to stimulate TNF was no longer present in the fraction.

TABLE 4
Effect of F2 and F3 on cytokine levels in mice administered
anti-CD3 mAb

| | TNF (2 hrs) | IL-2 (2 hrs) | IFNγ (6 hrs) |
|---|---|---|---|
| Saline | 236 ± 17 | 398 ± 43 | 269 ± 56 |
| F2 and F3 | 218 ± 10 | 224 ± 21 | 199 ± 22 |
| % reduction | <10% | 44%* | 26% |

*p < 0.05
**F2 and F3 were combined in the same relative proportions obtained in natural bromelain (85:15).

Combined fraction F2&F3 of bromelain was expected to have inhibitory activity, as it contains the F3 fraction, and ananain the immunosuppressive component. So, it was surprising that it did not have an inhibitory effect against TNF. The reduced effectiveness against IFNγ and nil effect against TNF may be because only small amounts of F3 or ananain are present in the F2&F3 fraction. That is the F2&F3 fraction used contained the relative proportions usually found in natural bromelain (ratio of F2:F3 of 85:15), That is the F3 fraction only comprised 15% of the total F2&F3 traction.

An F2/F3 fraction that has a higher proportion of F3 (and therefore ananain) to F2 would therefore be expected to have a greater immunosuppressive effect.

### Effect of New F2&F3 Mixture on Cytokine Levels In Vivo

We next investigated the effect of the new F2&F3 mixture, that contained a higher proportion of F3 to F2 (and minus the F1 fraction), on cytokine levels in vivo (n=6 animals per group). We investigated its effect against TNF, which as mentioned previously is a potent inflammatory cytokine. In addition, we investigated its effect against monocyte chemoattractant protein-1 (MCP-1/CCL2), a key inflammatory chemokine implicated in the pathogenesis of several inflammatory disorders.

Table 5 shows that at 2 and 6 hours post antibody administration, the new F2&F3 mixture decreased TNF and MCP-1 levels by 20%. In contrast, F2 and F3 either had minimal effect (as seen earlier in Table 4 or actually increased cytokine production. F3, as expected, reduced TNF and MCP-1 by 35% and 40% respectively.

TABLE 5
Effect of F3, combined F2 and F3 and the new F2&F3 mixture on
cytokine levels in mice administered anti-CD3 mAb

| | TNF (2 hrs) | % reduction | MCP-1 (6 hrs) | % reduction |
|---|---|---|---|---|
| Saline | 187 ± 15 | — | 3,877 ± 269 | — |
| F3 | 122 ± 48 | 35% | 2,223 ± 1131 | 40% |
| F2 and F3 | 179 ± 20 | 4% | 3,754 ± 271 | Incr by 8% |
| New | 155 ± 16 | 20% | 3,491 ± 254 | 20% |
| F2&F3 | | | | |

One way to efficiently produce F2:F3 fractions with different proportions of active F3, would be to express the active ananain in an expression system, such as Pichia pastoris. P. pastoris is frequently used to express recombinant proteins. It has a high growth rate and is able to grow on inexpensive mediums on a large scale in commercial fermenters.

F2 and F3 expressing clones could be produced separately in different fermenters and re-constituted in the desired proportions, or can be co-expressed at the same time in the one fermenter.

In summary, F3 inhibits cytokine production, while F1 has the opposite effect to stimulate cytokines, while F2 had no effect, reflecting the different biological activities of the components within the bromelain fractions tested.

In two studies of the dextran sodium sulphate (DSS)-induced murine model of ulcerative colitis (inflammatory bowel disease), bromelain, CCS and ananain met the primary end point of increasing colon length compared to a vehicle control group. The first of these studies was a prophylactic study and the second was a treatment study. There was also significant improvement in other clinical indicators of disease, including reduced colorectal bleeding, increased body weight and reduced diarrhea. This model is widely regarded to reproduce many of the clinical features of human inflammatory bowel disease, particularly colitis, and is considered a very difficult model to demonstrate therapeutic effects. Bromelain and ananain also decreased production of a number of key inflammatory cytokines, including IFNγ and TNF.

Taken together, these data indicate that the several different components of crude bromelain in fact have differing activities. Anti-secretory activity is produced by each of two different proteases, identifiable in ion exchange chromatography as peaks “CCY” and “CCW.” Both of these peaks occur in Fraction F2, and can thus be considered part of the “stem bromelain” crude extract.

In contrast, anti-inflammatory activity is produced by ananain, identifiable as the “CCS”

peak occurring in Fraction F3 in ion exchange chromatography separation.

| Description | Anti-secretory active | Anti-inflammatory active |
|---|---|---|
| Fraction | F2 | F3 |
| Peak | CCY & CCW | CCS |
| Pure API | Stern bromelain protease | ananain |

In contrast, in my tests, Fraction F1 did not appear to provide any anti-inflammatory activity, and may interfere with the remaining fractions' anti-inflammatory effect.

### The Use of F2&F3, Stem Bromelain Protease & Ananain to Prevent Inflammation at Weaning, to Improve Gut Health, and Increase Feed Intake

Given the different activities I have identified in the different fractions, one can improve prior art bromelain products by using not the whole crude bromelain extract, but a specific fraction of it, i.e., Fraction F2 or Fraction F3, or alternatively whole crude extract with Fraction F1 removed. More specifically, one may use the CCS portion of Fraction F3, and more specifically, one may use purified ananain.

Fraction F2, or Fraction F3, or whole crude extract with Fraction F1 removed, or the CCS portion of Fraction F3, or purified ananain may be used in lieu of crude bromelain extract to provide a dosage suitable for prophylactic treatment of a suckling piglet to prevent scour. Use to treat (rather than prevent) scour may require a different dose; the artisan would readily be able to derive the appropriate dose. Similarly, use to treat a mature adult pig, or a human may require a larger dose; the artisan would readily be able to derive the appropriate dose. The amount of active (Fraction F2 or Fraction F3, or whole crude extract with Fraction F1 removed, or the CCS portion of Fraction F3, or purified ananain.) required depends on the specific kind of active (one needs less mass of purified ananain than of Fraction 3) and the activity of that enzyme(s), but should in any event be a smaller mass than required for an equivalent enzyme activity of crude bromelain extract.

One may provide my formulation as an oral drench, for example as a granulated powder requiring reconstitution with water. To prevent post-weaning scour, my formulation may be given as a once only oral dose on the day of weaning (1-2 days before the expected on set of scour). To prevent pre-weaning scour, a single oral dose can be administered at 2-5 days of age, depending on a particular farm's problem period. A repeat dose may be required 3-7 days later. As a treatment, my formulation may be administered immediately when symptoms of disease occur.

One may provide my formulation as a feed additive, for example prepared as a granulated powder that can be added to pig feed. To ensure thorough dispersion of the product it should first be mixed with a suitable quantity of feed ingredients before incorporation in the final mix. My formulation may be fed as a pre-mix only, or the pre-mix incorporated in the final mix. The recommended dose level provides at least equivalent enzymatic activity to that used in prior art whole bromelain compositions, and may be fed daily for roughly 14 consecutive days, or added to water.

Alternatively, my formulation may be provided as tablet and capsules, and other appropriate dose forms for humans.

The skilled artisan may adjust my formulation for different indications. For example, it may be used for the prevention and treatment of scour in production animals (cattle, swine etc.) and diarrhea in humans. It may also be used for improved gut health by reducing inflammation. Alternatively, it may be formulated to promote increased feed intake in production animals, thus promoting weight gains and feed conversion efficiency. It may be used to reduce the requirement for antibiotics in animal feed, and for acute administration to humans. It may also be used to ameliorate Inflammatory Bowel Disease in humans.

### SUMMARY

Given my disclosure here, one may readily make certain variants and alternatives. I thus intend the legal coverage of this patent to be defined not by the specific example recited here, but by the legal claims and permissible equivalents thereof.

1.-13. (canceled)

14. A bromelain extract comprising stem bromelain protease and ananain in a w/w ratio of less than 2:1.

15. A bromelain extract of claim 14 comprising stem bromelain protease and ananain in a w/w ratio of less than 1:1.

16. A bromelain extract of claim 14 comprising stem bromelain protease and ananain in a w/w ratio of less than 0.5:1.

17. A bromelain extract of claim 14 comprising stem bromelain protease and ananain in a w/w ratio of less than 0.25:1.

18. A bromelain extract of claim 14 comprising stem bromelain protease and ananain in a w/w ratio of about 0.23:1.

19. A process of producing a bromelain extract comprising:
providing a crude bromelain;
separating components of the crude bromelain into distinct eluents by fast flow high performance liquid chromatography on S-Sepharose eluting with a gradient of about 0 to about 1 M NaCl in buffer, wherein distinct eluents correspond to peaks 1-9 of FIG. 2A; and
obtaining eluents corresponding to peaks 5-9 shown in FIG. 2A.

20. The process of claim 19 wherein obtaining eluents corresponding to peaks 5-9 shown in FIG. 2A comprises:
collecting eluents corresponding to peaks 1-4 shown in FIG. 2A to produce a first fraction;
collecting eluents corresponding to peaks 5-7 shown in FIG. 2A to produce a second fraction,
collecting eluents corresponding to peaks 8-9 shown in FIG. 2A to produce a third fraction, and
combining the second fraction with the third fraction without combining the first fraction to provide the bromelain extract.

21. The process of claim 19 wherein the buffer comprises acetate buffer.

22. The process of claim 20 wherein the second fraction and the third fraction are in a w/w ratio of less than 2:1 in the bromelain extract.

23. The process of claim 20 wherein the second fraction and the third fraction are in a w/w ratio of less than 1:1 in the bromelain extract.

24. The process of claim 20 wherein the second fraction and the third fraction are in a w/w ratio of less than 0.5:1 in the bromelain extract.

25. The process of claim 20 wherein the second fraction and the third fraction are in a w/w ratio of less than 0.25:1 in the bromelain extract.

26. the process of claim 20 wherein the second fraction and the third fraction are in a w/w ratio of 0.23:1 in the bromelain extract.

27. A bromelain extract obtained from the process of claim 19.

28. A pharmaceutical composition comprising the bromelain extract of claim 27 and a pharmaceutically acceptable excipient.

29. A method of blocking production of one or more cytokines selected from the group comprising IL-2, IL-4, TNF and IFNγ in a subject in need thereof, the method comprising administering to the subject a pharmaceutical composition of claim 28.
